(19) United States
(12) Patent Application Publication
(10) Pub. No.: US 2024/0407714 A1
(43) Pub. Date: Dec. 12, 2024

(54) SYSTEM AND METHOD FOR USER DATA-BASED LOCATION MANAGEMENT

(71) Applicant: PLUME DESIGN, INC., Palo Alto, CA (US)

(72) Inventors: Derek LOWNSBROUGH, Kelowna (CA); Mark BREZNIK, Ljubljana (SI); Satish RAJASEKARAN, Newark, CA (US)

(21) Appl. No.: 18/329,620

(22) Filed: Jun. 6, 2023

(51) Int. Cl. A61B 5/00; G16H 50/20

(52) U.S. Cl. CPC A61B 5/4815; G16H 50/20

(57) ABSTRACT

Disclosed are systems and methods that provide a novel framework for personalized sleep management and control for a user. The framework can provide dynamically determined, customized sleep recommendations and/or optimized sleep configurations that enable users to engage in deeper, longer and more restful sleep, as specified to their specific needs. The framework provides a comprehensive sleep optimization system that delves deep into the factors that influence a user's sleep quality by measuring a range of sleep and lifestyle factors, and providing personalized insights and recommendations to aid and/or effectuate users achieving a restful and rejuvenating sleep.

### FIELD OF THE DISCLOSURE

The present disclosure is generally related to a sleep monitoring and control system, and more particularly, to a decision intelligence (DI)-based computerized framework for automatically and dynamically managing real-time sleep activity of a user.

### BACKGROUND

In today's fast-paced, technology-driven world, many individuals struggle to understand how daily activities, screen time and network activity, inter alia, such as gaming and streaming, impact their sleep quality. Indeed, conventional mechanisms for providing sleep management functionality fall short of providing the accurate, efficient and optimized sleep control and/or management that many modern people desperately need.

### SUMMARY OF THE DISCLOSURE

Moreover, a lack of personalized, data-driven insights makes it challenging for individuals to make informed decisions about the ideal amount of non-screen time before bedtime, tailored specifically to their needs. To that end, according to some embodiments, the disclosed systems and methods provide a novel computerized framework that addresses such current shortcomings, among others, and provides dynamically determined, personalized sleep recommendations and/or optimized sleep configurations that enable users to engage in deeper, longer and more restful sleep, as customized to their specific needs.

According to some embodiments, as discussed herein, the disclosed systems and methods provide a comprehensive sleep optimization system that delves deep into the factors that influence a user's sleep quality. In some embodiments, by measuring a range of sleep and lifestyle factors, as discussed below, the disclosed framework can provide personalized insights and recommendations to assist and/or effectuate users achieving a restful and rejuvenating sleep.

According to some embodiments, as discussed herein, the disclosed framework can collect data about a user, which can be from any device associated with a user as well as from a variety of data resources (e.g., cloud-hosted data, for example), as discussed in more detail below in relation to at least FIGS. 1 and 3-4. For example, data collected from a user's smart ring, smart phone, sensors at a location and/or network-based data stored in a cloud can provide the data from which the disclosed framework can learn and/or determine/develop a personalized pattern (or circadian rhythm) which can be utilized to recommend sleep factors (e.g., real-world and/or digital) to the user.

By way of a non-limiting example, according to some embodiments, the disclosed framework can collect data, which can include measurement values related to, but not limited to, sleep latency, deep sleep duration, light sleep duration, restlessness, nighttime awakenings, screen usage during and/or proximate to sleep time, digital activity, real-world activities, demographics, location information (e.g., light wattage in room, for example), network data/traffic, and the like, or some combination thereof. Such data can be analyzed and the determinations performed therefrom can be leveraged to personalize sleep recommendations and/or sleep management optimizations for the user.

According to some embodiments, a method is disclosed for a DI-based computerized framework for automatically and dynamically managing real-time sleep activity of a user. In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform a method for automatically and dynamically managing real-time sleep activity of a user.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary workflow according to some embodiments of the present disclosure;

FIG. 4 illustrates an exemplary workflow according to some embodiments of the present disclosure;

FIG. 5 depicts an exemplary implementation of an architecture according to some embodiments of the present disclosure;

FIG. 6 depicts an exemplary implementation of an architecture according to some embodiments of the present disclosure; and

FIG. 7 is a block diagram illustrating a computing device showing an example of a client or server device used in various embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2^{nd}, 3^{rd}, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

Certain embodiments and principles will be discussed in more detail with reference to the figures. According to some embodiments, as discussed in more detail below, the disclosed framework can function/operate to provide personalized insights and/or recommendations to users based on their own specific/unique data. Thus, a tailored approach to sleep improvement can be compiled and automatically provided to the user, which can be provided upon request and/or a determined time a user is deemed to be about to sleep. In some embodiments, the disclosed framework can leverage such insights, in addition to the recommendations to the users, to control the location in which they are sleeping. For example, access points, devices (e.g., televisions, smart phones, and the like), lights and/or any other smart home feature can be engaged and/or toggled to different and/or modified modes upon the determination of a user's sleep, which can assist the user from engaging in activity that may be detrimental to their known sleep habits.

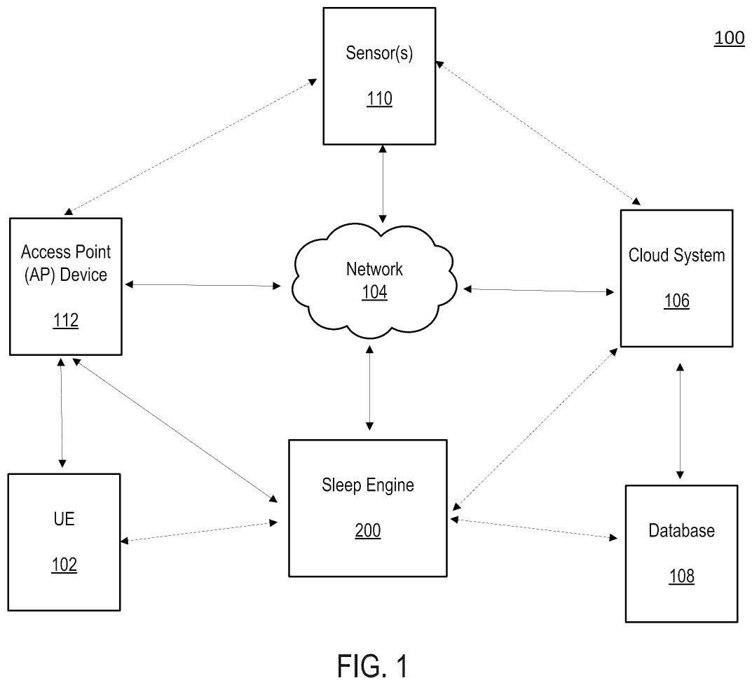

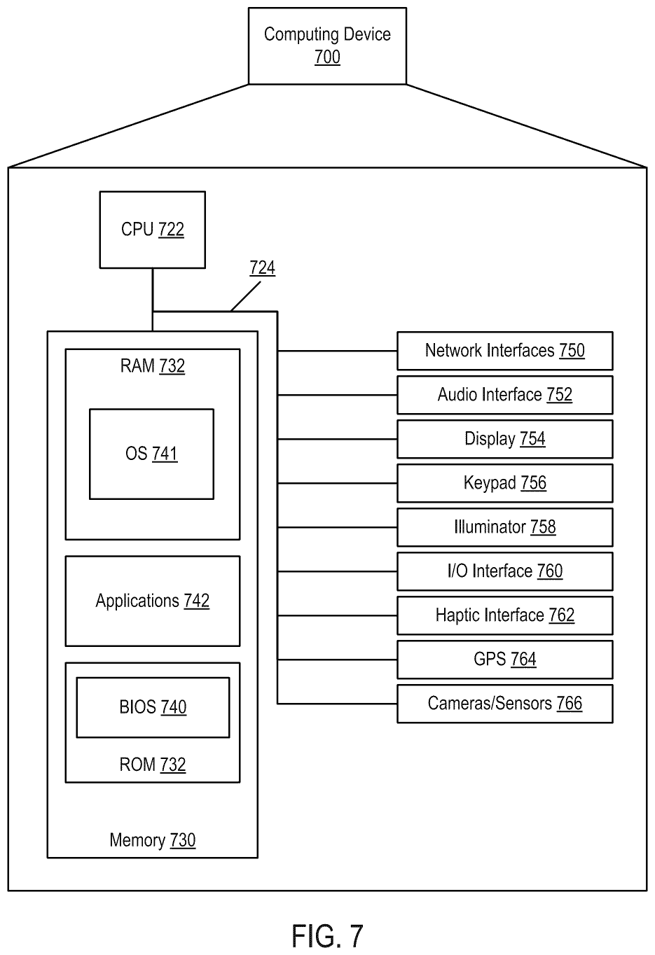

With reference to FIG. 1, system 100 is depicted which includes user equipment (UE) 102 (e.g., a client device, as mentioned above and discussed below in relation to FIG. 7), access point (AP) device 112, network 104, cloud system 106, database 108, sensors 110 and sleep engine 200. It should be understood that while system 100 is depicted as including such components, it should not be construed as limiting, as one of ordinary skill in the art would readily understand that varying numbers of UEs, AP devices, peripheral devices, sensors, cloud systems, databases and networks can be utilized; however, for purposes of explanation, system 100 is discussed in relation to the example depiction in FIG. 1.

According to some embodiments, UE 102 can be any type of device, such as, but not limited to, a mobile phone, tablet, laptop, sensor, IoT device, autonomous machine, and any other device equipped with a cellular or wireless or wired transceiver. For example, UE 102 can be a smart ring, which as discussed below in more detail, can enable the identification and/or collection of vitals of the wearing user. In some embodiments, such vitals can correspond to, but not be limited to, heart rate, heart rate variability (HRV), blood oxygen levels, blood pressure, hydration temperature, pulse, motion, sleep, and/or any other type of biometric for a person, or some combination thereof.

In some embodiments, peripheral device (not shown) can be connected to UE 102, and can be any type of peripheral device, such as, but not limited to, a wearable device (e.g., smart ring or smart watch), printer, speaker, sensor, and the like. In some embodiments, peripheral device can be any type of device that is connectable to UE 102 via any type of known or to be known pairing mechanism, including, but not limited to, WiFi, Bluetooth™, Bluetooth Low Energy (BLE), NFC, and the like. For example, the peripheral device can be a smart ring that connectively pairs with UE 102, which is a user's smart phone.

According to some embodiments, AP device 112 is a device that creates a wireless local area network (WLAN) for the location. According to some embodiments, the AP device 112 can be, but is not limited to, a router, switch, hub and/or any other type of network hardware that can project a WiFi signal to a designated area. For example, an AP device 112 can be a Plume Pod™ and the like. In some embodiments, UE 102 may be an AP device.

According to some embodiments, sensors 110 (or sensor devices 110) can correspond to any type of device, component and/or sensor associated with a location of system 100 (referred to, collectively, as “sensors”). In some embodiments, the sensors 110 can be any type of device that is capable of sensing and capturing data/metadata related to a user and/or activity of the location. For example, the sensors 110 can include, but not be limited to, cameras, motion detectors, door and window contacts, heat and smoke detectors, passive infrared (PIR) sensors, time-of-flight (ToF) sensors, and the like.

In some embodiments, the sensors 110 can be associated with devices associated with the location of system 100, such as, for example, lights, smart locks, garage doors, smart appliances (e.g., thermostat, refrigerator, television, personal assistants (e.g., Alexa®, Nest®, for example)), smart rings, smart phones, smart watches or other wearables, tablets, personal computers, and the like, and some combination thereof. For example, the sensors 110 can include the sensors on UE 102 (e.g., smart phone) and/or peripheral device (e.g., a paired smart watch). In another example, sensors 110 can correspond to the sensors on a user's smart ring.

In some embodiments, network 104 can be any type of network, such as, but not limited to, a wireless network, cellular network, the Internet, and the like (as discussed above). Network 104 facilitates connectivity of the components of system 100, as illustrated in FIG. 1.

According to some embodiments, cloud system 106 may be any type of cloud operating platform and/or network based system upon which applications, operations, and/or other forms of network resources may be located. For example, system 106 may be a service provider and/or network provider from where services and/or applications may be accessed, sourced or executed from. For example, system 106 can represent the cloud-based architecture associated with a smart home or network provider, which has associated network resources hosted on the internet or private network (e.g., network 104), which enables (via engine 200) the sleep management discussed herein.

In some embodiments, cloud system 106 may include a server(s) and/or a database of information which is accessible over network 104. In some embodiments, a database 108 of cloud system 106 may store a dataset of data and metadata associated with local and/or network information related to a user(s) of the components of system 100 and/or each of the components of system 100 (e.g., UE 102, AP device 112, sensors 110, and the services and applications provided by cloud system 106 and/or sleep engine 200).

In some embodiments, for example, cloud system 106 can provide a private/proprietary management platform, whereby engine 200, discussed infra, corresponds to the novel functionality system 106 enables, hosts and provides to a network 104 and other devices/platforms operating thereon.

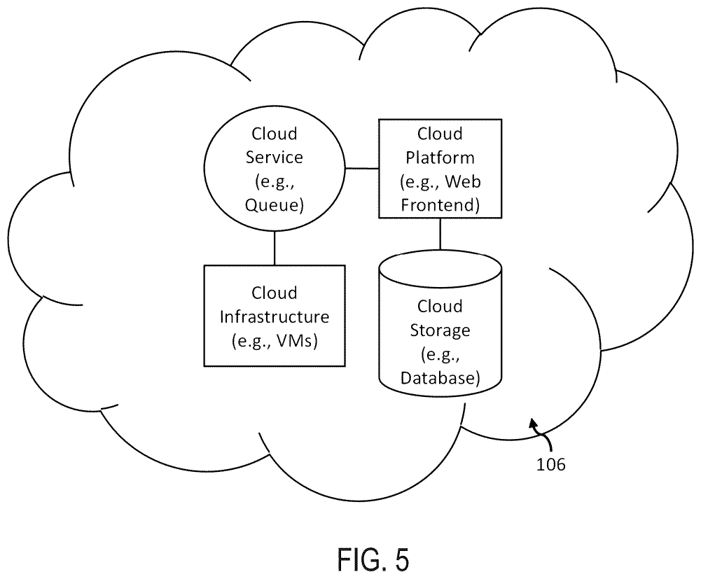

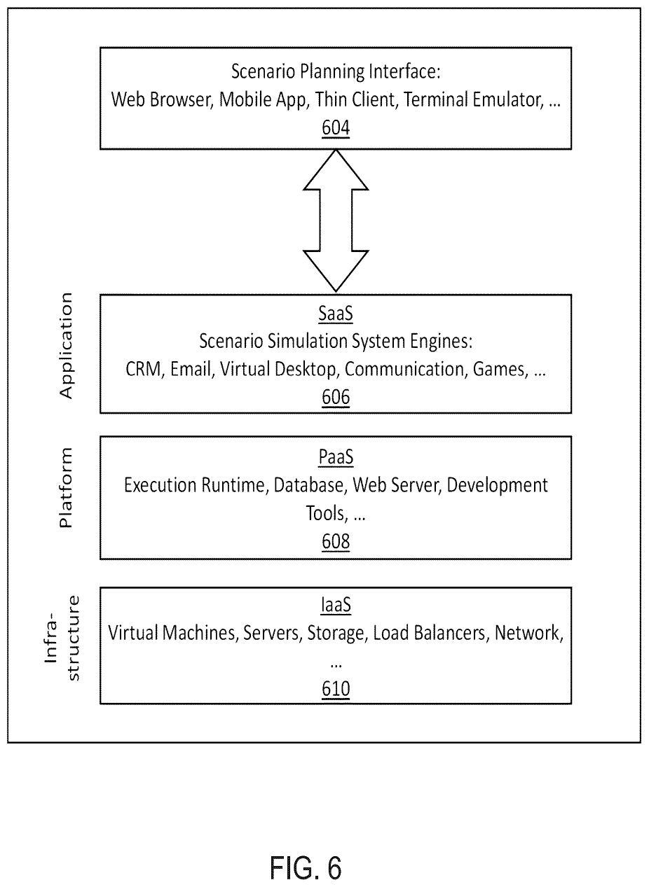

Turning to FIGS. 5 and 6, in some embodiments, the exemplary computer-based systems/platforms, the exemplary computer-based devices, and/or the exemplary computer-based components of the present disclosure may be specifically configured to operate in a cloud computing/architecture 120 such as, but not limiting to: infrastructure as a service (IaaS) 610, platform as a service (PaaS) 608, and/or software as a service (SaaS) 606 using a web browser, mobile app, thin client, terminal emulator or other endpoint 604. FIGS. 5 and 6 illustrate schematics of non-limiting implementations of the cloud computing/architecture(s) in which the exemplary computer-based systems for administrative customizations and control of network-hosted application program interfaces (APIs) of the present disclosure may be specifically configured to operate.

Turning back to FIG. 1, according to some embodiments, database 108 may correspond to a data storage for a platform (e.g., a network hosted platform, such as cloud system 106, as discussed supra) or a plurality of platforms. Database 108 may receive storage instructions/requests from, for example, engine 200 (and associated microservices), which may be in any type of known or to be known format, such as, for example, standard query language (SQL). According to some embodiments, database 108 may correspond to any type of known or to be known storage, for example, a memory or memory stack of a device, a distributed ledger of a distributed network (e.g., blockchain, for example), a look-up table (LUT), and/or any other type of secure data repository

Sleep engine 200, as discussed above and further below in more detail, can include components for the disclosed functionality. According to some embodiments, sleep engine 200 may be a special purpose machine or processor, and can be hosted by a device on network 104, within cloud system 106, on AP device 112 and/or on UE 102. In some embodiments, engine 200 may be hosted by a server and/or set of servers associated with cloud system 106.

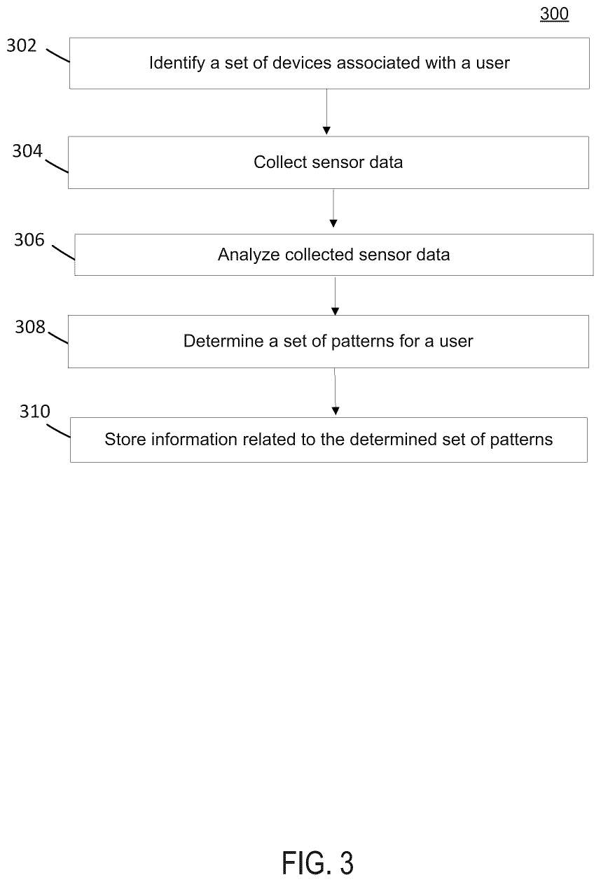

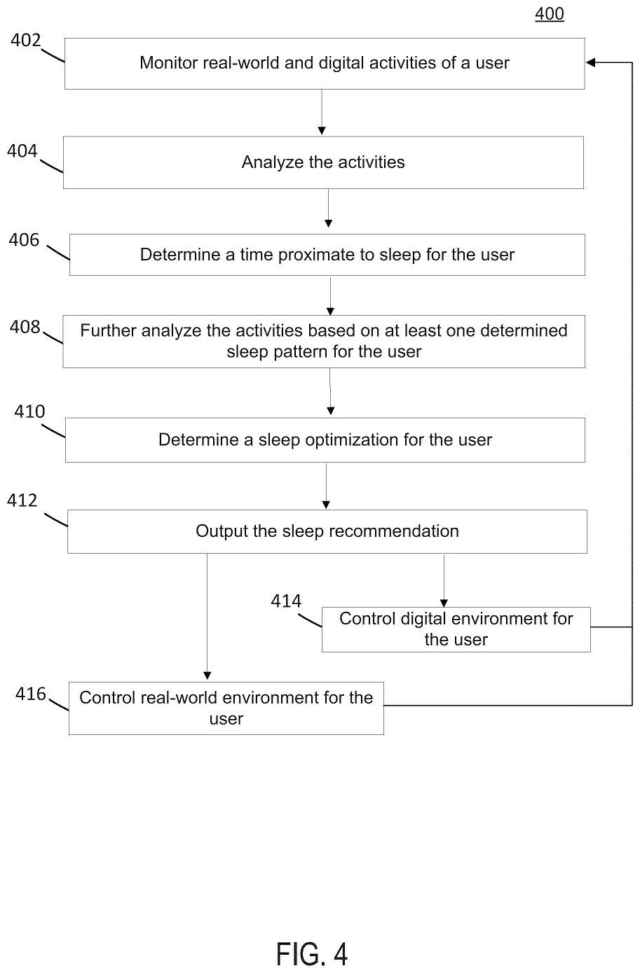

According to some embodiments, as discussed in more detail below, sleep engine 200 may be configured to implement and/or control a plurality of services and/or microservices, where each of the plurality of services/microservices are configured to execute a plurality of workflows associated with performing the disclosed security management. Non-limiting embodiments of such workflows are provided below in relation to at least FIGS. 3-4.

According to some embodiments, as discussed above, sleep engine 200 may function as an application provided by cloud system 106. In some embodiments, engine 200 may function as an application installed on a server(s), network location and/or other type of network resource associated with system 106. In some embodiments, engine 200 may function as an application installed and/or executing on UE 102 and/or sensors 110 (and/or AP device 112, in some embodiments). In some embodiments, such application may be a web-based application accessed by AP device 112, UE 102 and/or devices associated with sensors 110 over network 104 from cloud system 106. In some embodiments, engine 200 may be configured and/or installed as an augmenting script, program or application (e.g., a plug-in or extension) to another application or program provided by cloud system 106 and/or executing on AP device 112, UE 102 and/or sensors 110.

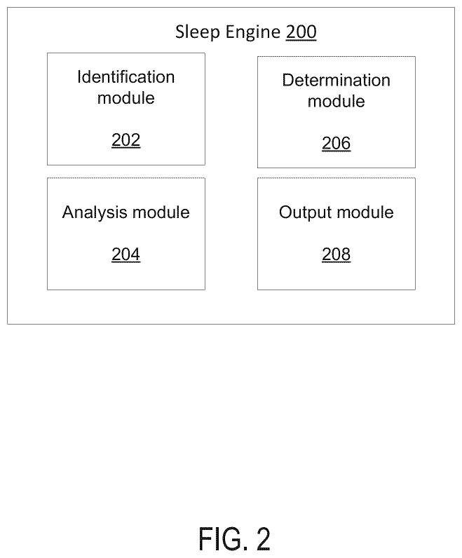

As illustrated in FIG. 2, according to some embodiments, sleep engine 200 includes identification module 202, analysis module 204, determination module 206 and output module 208. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

Turning to FIG. 3, Process 300 provides non-limiting example embodiments for the disclosed security management framework. According to some embodiments, Process 300 provides non-limiting embodiments for determining user information and corresponding patterns of behavior for which the disclosed framework (e.g., via sleep engine 200) can control, manage and manipulate the operational status of a location. As discussed below at least in relation to Process 400 of FIG. 4, the leveraged and stored information can be utilized to optimize a user's sleep.

According to some embodiments, Steps 302-304 of Process 300 can be performed by identification module 202 of sleep engine 200; Step 306 can be performed by analysis module 204; Step 308 can be performed by determination module 204; and Step 310 can be performed by output module 208.

According to some embodiments, Process 300 begins with Step 302 where a set of devices associated with a user (and in some embodiments, associated with a location, for example, a user's home) are identified. According to some embodiments, the devices can be associated with any of type of UE 102, AP device 112, sensors 110, and the like, discussed above in relation to FIG. 1. For example, the devices can at least include, but are not limited to, a user's smart ring that they wear daily, their smart phone, tablet devices in the home connected to the local network (e.g., Wi-Fi), televisions, light sensors, door sensors, routers/modems providing the network for the location, and the like. Additional, non-limiting examples of sensors and the types of collectable data are discussed above at least in relation to FIG. 1.

In some embodiments, the identified devices can be paired and/or connected with another device (e.g., sensor 110, engine 200 and/or UE 102) via a cloud (e.g., Plume® cloud, for example) and/or cloud-to-cloud (C2C) connection (e.g., establish connection with a third party cloud, which connects with cloud system 106, for example).

In Step 304, engine 200 can operate to trigger the identified devices to collect data about the user (e.g., referred to as user data). According to some embodiments, the user data can be collected continuously and/or according to a predetermined period of time or interval. In some embodiments, user data may be collected based on detected events. In some embodiments, type and/or quantity of user data may be directly tied to the type of device performing such data collection. For example, a sensor associated with lights within the user's bedroom may only collect user data when a light in that room is turned off (and/or turned off from previously being turned on). For example, such data can provide, but is not limited to, an on event, which can indicate, but is not limited to, the identity of the light/room, time of toggling, duration of being turned on, frequency of being turned on, and the like, or some combination thereof). In another non-limiting example, a gyroscope sensor on a user's smartphone and/or smart ring can detect when a user is moving, the type and/or metrics of such movements.

According to some embodiments, the user data can be derived from data collected from UE 102 (e.g., smart ring). As discussed above, the user data can provide biometrics or vitals for the user, which can include information related to, but are not limited to, heart rate, HRV, blood oxygen levels, blood pressure, hydration temperature, pulse, motion, sleep, and the like, or some combination thereof.

Accordingly, in some embodiments, the vitals, inclusive of the heart rate, HRV, blood pressure and blood oxygen level information, for example, can provide insights into a user's cardiovascular health, respiratory health and fitness level. In some embodiments, the vitals can further provide tracked information related to the user's physical activity, including, but not limited to, the number of steps taken, distance traveled, and calories burned, and the like. In some embodiments, the vitals can also include information related to a user's stress levels, which can include data related to the user's stress levels, for example. In some embodiments, the vitals can further provide data corresponding to the user's body temperature, which can provide, for example, information about their overall health and detect potential illnesses.

In some embodiments, such vitals can further provide information and/or measurement (values or metrics) related to their sleep data, which can include, but is not limited to, sleep latency (e.g., time it takes for the user to fall asleep or into a deep sleep, and the like), deep sleep duration (e.g., length of time spent in restorative sleep), light sleep duration (e.g., time spent in lighter sleep stages), restlessness (e.g., frequency and/or degree of movement during sleep and/or when lying in bed), nighttime awakenings (e.g., number of times the user wakes up and/or gets out of bed), screen usage during sleep time (e.g., amount of time a smart phone or related device is being used by the when they are lying in bed and/or in the bedroom), and the like or some combination thereof.

In some embodiments, the user data can further or alternatively be based on lifestyle factors, which can include, but are not limited to, screen/Internet use before bed (e.g., time spent on screens before bed throughout the day), type of network activity (e.g., gaming, social media, streaming, and the like), exercise patterns (e.g., timing, duration, and/or intensity of physical activity during the day), motion detection and/or user location (e.g., motion data in/around and/or outside the location during the day and/or around bedtime), diet and nutrition, and the like, or some combination thereof.

In some embodiments, such user data may be derived and/or mined from stored user data within an associated or third party cloud. For example, engine 200 can be associated with a cloud, which can store collected network traffic and/or collected user data for the user in an associated account of the user. Thus, in some embodiments, Step 304 can involve querying the cloud for information about the user, which can be based on a criteria that can include, but is not limited to, a time, date, activity, event, other collected user data, and the like, or some combination thereof.

In some embodiments, the collected user data in Step 304 can be stored in database 108 in association with an identifier (ID) of a user, an ID of the location and/or an ID of an account of the user/location.

In Step 306, engine 200 can analyze the collected user data. According to some embodiments, engine 200 can implement any type of known or to be known computational analysis technique, algorithm, mechanism or technology to analyze the collected user data from Step 306.

In some embodiments, engine 200 may include a specific trained artificial intelligence/machine learning model (AI/ML), a particular machine learning model architecture, a particular machine learning model type (e.g., convolutional neural network (CNN), recurrent neural network (RNN), autoencoder, support vector machine (SVM), and the like), or any other suitable definition of a machine learning model or any suitable combination thereof.

In some embodiments, engine 200 may be configured to utilize one or more AI/ML techniques chosen from, but not limited to, computer vision, feature vector analysis, decision trees, boosting, support-vector machines, neural networks, nearest neighbor algorithms, Naive Bayes, bagging, random forests, logistic regression, and the like. By way of a non-limiting example, engine 200 can implement an XGBoost algorithm for regression and/or classification to analyze the user data, as discussed herein.

According to some embodiments, the AI/ML computational analysis algorithms implemented can be applied and/or executed in a time-based manner, in that collected user data for specific time periods can be allocated to such time periods so as to determine patterns of activity (or non-activity) according to a criteria. For example, engine 200 can execute a Bayesian determination for a predetermined time span, at preset intervals (e.g., a 24 hour time span, every 8hours, for example), so as to segment the day according to applicable patterns, which can be leveraged to determine, derive, extract or otherwise activities/non-activities in/around a location.

In another example, the patterns can correspond to portions of the day that correspond to nighttime or sleep time (e.g., which can be based on sunrise and/or sunset at the user's location).

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define Neural Network architecture/model,
b. transfer the input data to the neural network model,
c. train the model incrementally,
d. determine the accuracy for a specific number of timesteps,
c. apply the trained model to process the newly-received input data,
f. optionally and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

In Step 308, based on the analysis from Step 306, engine 200 can determine a set of patterns for a user(s) (and/or patterns for the location). According to some embodiments, the determined patterns are based on the computational AI/ML analysis performed via engine 200, as discussed above.

In some embodiments, the set of patterns can correspond to, but are not limited to, types of events, types of detected activity, a time of day, a date, type of user, duration, amount of activity, quantity of activities, sublocations within the location (e.g., rooms in the house, for example), and the like, or some combination thereof. Accordingly, the patterns can be specific to a user, and/or specific to the location (e.g., or room within a location-for example, the bedroom of the location).

Thus, according to some embodiments, Step 308 can involve engine 200 determining a set of sleep patterns and/or relationships to sleep activity of the user based on the user data, which as discussed below at least in relation to Process 400 of FIG. 4, can be utilized to generate personalized sleep recommendations and/or controls for the user.

In Step 310, engine 200 can store the determined set of patterns in database 108, in a similar manner as discussed above. According to some embodiments, Step 310 can involve creating a data structure associated with each determined pattern, whereby each data structure can be stored in a proper storage location associated with an identifier of the user/location, as discussed above.

In some embodiments, a pattern can comprise a set of events, which can correspond to an activity and/or non-activity (e.g., exercising in the house, cleaning the dishes, sleeping, and the like, for example). In some embodiments, the pattern's data structure can be configured with header (or metadata) that identifies a user and/or the location, and/or a time period/interval of analysis (as discussed above); and the remaining portion of the structure providing the data of the activity/non-activity and status of entry-points during such sequence(s). In some embodiments, the data structure for a pattern can be relational, in that the events of a pattern can be sequentially ordered, and/or weighted so that the order corresponds to events with more or less activity.

In some embodiments, the structure of the data structure for a pattern can enable a more computationally efficient (e.g., faster) search of the pattern to determine if later detected events correspond to the events of the pattern, as discussed below in relation to at least Process 400 of FIG. 4. In some embodiments, the data structures of patterns can be, but are not limited to, files, arrays, lists, binary, heaps, hashes, tables, trees, and the like, and/or any other type of known or to be known tangible, storable digital asset, item and/or object.

According to some embodiments, the user data can be identified and analyzed in a raw format, whereby upon a determination of the pattern, the data can be compiled into refined data (e.g., a format capable of being stored in and read from database 108). Thus, in some embodiments, Step 310 can involve the creation and/or modification (e.g., transformation) of the user data into a storable format.

In some embodiments, as discussed below, each pattern (and corresponding data structure) can be modified based on further detected behavior, as discussed below in relation to Process 400 of FIG. 4.

Turning to FIG. 4, Process 400 provides non-limiting example embodiments for the deployment and/or implementation of the disclosed sleep management framework.

By way of non-limiting example, upon a determination that a user is about to go to sleep (e.g., a known time the user typically goes to sleep is detected and/or the lights in their bedroom are turned off and it is detected the user is laying in his/her bed, for example), engine 200 can provide sleep recommendations to the user and/or automatically operate to control the sleep environment of the user. For example, an application executing on the user's smart phone can provide a notification that can include information indicating suggestions for their sleep (e.g., turn off phone, turn on fan in the room, stop or do not eat further, and the like). In another example, engine 200 can automatically act to throttle network traffic which can render the user's smart phone incapable of accessing network resources, for example.

According to some embodiments, Step 402 can be performed by identification module 202 of sleep engine 200; Steps 404 and 408 can be performed by analysis module 204; Steps 406 and 410 can be performed by determination module 206; and Steps 412-416 can be performed by output module 208.

According to some embodiments, Process 400 begins with Step 402 where engine 200 can monitor the location to detect, determine or otherwise identify activity related to user movement (or non-movement) at the location. In some embodiments, engine 200 can monitor the location continuously, and/or according to a predetermined time interval. In some embodiments, the monitoring of the location can be performed via the devices identified in Step 302, discussed supra. In some embodiments, the monitoring can involve periodically pinging each or a portion of the devices at the location, and awaiting a reply. In some embodiments, the monitoring can involve push and/or fetch protocols to collect user data from each sensor.

In Step 404, based on the monitoring of the location, engine 200 can analyze the monitored activities (and corresponding collected data based therefrom), which can be performed in a similar manner as discussed above at least in relation to Step 306. In some embodiments, the collected activity data from Step 402 can be mined and/or parsed for data related to sleep activity of the user, whereby such sleep activity data can be extracted and analyzed therefrom.

In Step 406, engine 200 can determine a time proximate to sleep for the user. In some embodiments, such determination can be based on analysis and/or retrieval of information from the stored sleep partners, as discussed above in relation to Step 310. In some embodiments, such determination can be based on analysis of the collected activity data from Step 404, whereby activities indicating the user is laying in his/her bed can be derived based on the AI/ML analysis performed in Step 404. In some embodiments, the collected data (from Step 404) can be analyzed in connection with retrieved stored pattern data (from Step 310), whereby the determination as to when the user will be going to sleep can be performed.

In some embodiments, a time proximate the user sleeps can correspond to a range of time related to the predicted time the user can/will go to bed. For example, such range can be a predetermined number, for example, 30 minutes. Therefore, the determination can involve identifying when the user is predicted to go to sleep, and the time proximate can be 30 minutes prior. Thus, as provided below, the time proximate can be utilized as a time to provide sleep recommendations and/or controls for the user, as discussed at least in relation to Step 412, discussed infra.

In some embodiments, in Step 408, engine 200 can compare the activities from Step 404 to the stored sleep patterns (from Step 310), as discussed above. In some embodiments, Step 408 may be by-passed when such analysis is performed as part of Step 404, as discussed above. In some embodiments, Step 408 can be utilized to confirm and/or verify a prediction that is specific to the user. For example, a pattern of activity for a weekend can be utilized to perform a prediction for that day, whereas a pattern of activity for a weekday can alternatively be used when the day is a weekday, and the like.

Accordingly, in Step 410, engine 200 can determine and/or compile a sleep optimization recommendation for the user. The sleep optimization recommendation (or sleep recommendation, used interchangeably), can provide a set of instructions that can be implemented and/or executed so as to enable the user to engage in a more restful sleep, as discussed above. For example, the sleep recommendation can include information related to, but not limited to, screen time limits, Internet traffic limits, television limits, preferred temperatures in the location, lighting instructions, recommendations for amount of blankets on the bed as per the user's ideal sleep temperature (against the indoor temperature and outside climate), amount of pillows (e.g., head or leg angle to induce ideal blood flow for the user during sleep, a time to cut off eating, a preferred time to shower, and the like, or some combination thereof.

In some embodiments, such recommendation can be compiled as a data structure and stored in database 108, in a similar manner as discussed above. In some embodiments, such recommendation can be utilized to update the previously compiled, stored and utilized sleep pattern, as per Steps 406 and/or 408, discussed supra.

In Step 412, engine 200 can cause the sleep recommendation to output to the user. For example, the sleep recommendation can be displayed on a device of the user, which can be in the form of an application notification, electronic message, and the like. In some embodiments, the sleep recommendation can visibly display a set of steps and/or instructions for the user to perform. In some embodiments, the sleep recommendation can visibly display steps/instructions that engine 200 will automatically control, and in some embodiments, can provide interactive features for the user to approve and/or decline such optimizations.

According to some embodiments, engine 200 can then proceed to perform Steps 414 and/or 416. In some embodiments, execution of Steps 414 and/or 416 can be performed automatically via engine 200's execution and/or based on input provided to and/or received from the user, as discussed above.

In Step 414, engine 200 can control the digital environment for the user. For example, as discussed above, network traffic, devices and/or other types of known or to be known electronic devices can be managed according to the sleep optimization recommendation.

In Step 416, engine 200 can control the real-world environment for the user. For example, as discussed above, lights, temperature and/or other types of known or to be known real-world environmental items can be managed according to the sleep optimization recommendation.

Accordingly, after Step 414 and/or Step 416, engine 200 may then continue monitoring the user and/or location. In some embodiments, the monitoring can continue running in the backend, while certain modules of engine 200 execute to optimize sleep for the user.

According to some embodiments, a user and/or location can have a dedicated engine 200 model so that the sleep protocols discussed herein can be specific to the events and patterns learned and detected for the user and/or at that location. In some embodiments, the model can be specific for a user or set of users (e.g., users that live at a certain location (e.g., a house).

FIG. 7 is a schematic diagram illustrating a client device showing an example embodiment of a client device that may be used within the present disclosure. Client device 700 may include many more or less components than those shown in FIG. 7. However, the components shown are sufficient to disclose an illustrative embodiment for implementing the present disclosure. Client device 700 may represent, for example, UE 102 discussed above at least in relation to FIG. 1.

As shown in the figure, in some embodiments, Client device 700 includes a processing unit (CPU) 722 in communication with a mass memory 730 via a bus 724. Client device 700 also includes a power supply 726, one or more network interfaces 750, an audio interface 752, a display 754, a keypad 756, an illuminator 758, an input/output interface 760, a haptic interface 762, an optional global positioning systems (GPS) receiver 764 and a camera(s) or other optical, thermal or electromagnetic sensors 766. Device 700 can include one camera/sensor 766, or a plurality of cameras/sensors 766, as understood by those of skill in the art. Power supply 726 provides power to Client device 700.

Client device 700 may optionally communicate with a base station (not shown), or directly with another computing device. In some embodiments, network interface 750 is sometimes known as a transceiver, transceiving device, or network interface card (NIC).

Audio interface 752 is arranged to produce and receive audio signals such as the sound of a human voice in some embodiments. Display 754 may be a liquid crystal display (LCD), gas plasma, light emitting diode (LED), or any other type of display used with a computing device. Display 754 may also include a touch sensitive screen arranged to receive input from an object such as a stylus or a digit from a human hand.

Keypad 756 may include any input device arranged to receive input from a user. Illuminator 758 may provide a status indication and/or provide light.

Client device 700 also includes input/output interface 760 for communicating with external. Input/output interface 760 can utilize one or more communication technologies, such as USB, infrared, Bluetooth™M, or the like in some embodiments. Haptic interface 762 is arranged to provide tactile feedback to a user of the client device.

Optional GPS transceiver 764 can determine the physical coordinates of Client device 700 on the surface of the Earth, which typically outputs a location as latitude and longitude values. GPS transceiver 764 can also employ other geo-positioning mechanisms, including, but not limited to, triangulation, assisted GPS (AGPS), E-OTD, CI, SAI, ETA, BSS or the like, to further determine the physical location of client device 700 on the surface of the Earth. In one embodiment, however, Client device may through other components, provide other information that may be employed to determine a physical location of the device, including for example, a MAC address, Internet Protocol (IP) address, or the like.

Mass memory 730 includes a RAM 732, a ROM 734, and other storage means. Mass memory 730 illustrates another example of computer storage media for storage of information such as computer readable instructions, data structures, program modules or other data. Mass memory 730 stores a basic input/output system (“BIOS”) 740 for controlling low-level operation of Client device 700. The mass memory also stores an operating system 741 for controlling the operation of Client device 700.

Memory 730 further includes one or more data stores, which can be utilized by Client device 700 to store, among other things, applications 742 and/or other information or data. For example, data stores may be employed to store information that describes various capabilities of Client device 700. The information may then be provided to another device based on any of a variety of events, including being sent as part of a header (e.g., index file of the HLS stream) during a communication, sent upon request, or the like. At least a portion of the capability information may also be stored on a disk drive or other storage medium (not shown) within Client device 700.

Applications 742 may include computer executable instructions which, when executed by Client device 700, transmit, receive, and/or otherwise process audio, video, images, and enable telecommunication with a server and/or another user of another client device. Applications 742 may further include a client that is configured to send, to receive, and/or to otherwise process gaming, goods/services and/or other forms of data, messages and content hosted and provided by the platform associated with engine 200 and its affiliates.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising:
collecting, by a device, data about a user, the user data comprising information related to activities of the user;
analyzing, by the device, the user data based on at least one pattern of activity of the user;
determining, by the device, a sleep optimization recommendation for the user, the sleep optimization recommendation comprising a set of instructions to be performed within a location of the user prior to the user going to sleep; and
communicating, over a network, a notification to the user, the notification comprising information related to the sleep optimization recommendation, the notification comprising executable operations for managing the sleep of the user at the location.

2. The method of claim 1, wherein the notification causes automatic control of electronic devices at the location, the automatic control causing the electronic devices to operate in a manner as directed by the set of instructions.

3. The method of claim 1, wherein the notification is visibly displayed upon communication, wherein the visible display provides visibly displayed indications for the user related to how the user is recommended to act based on the set of instructions.

4. The method of claim 1, wherein the notification corresponds to real-world and digital components of an environment of the location, the environment corresponding to a bedroom of the user at the location.

5. The method of claim 1, wherein the user data comprises information corresponding to the user that is sleep latency, deep sleep duration, light sleep duration, restlessness, nighttime awakenings, screen usage during sleep time, motion or activity data at the location, cloud-hosted data about the user, biometrics of the user, and network data related to a network at the location.

6. The method of claim 1, further comprising:
determining a time proximate the user going to sleep, the time proximate corresponding to a time period prior to the user sleeping at the location for which the set of instructions are to be performed, wherein the communication of the notification corresponds to the time proximate.

7. The method of claim 1, wherein the collected user data corresponds to data collected by the device in accordance with a plurality of locations.

8. The method of claim 1, further comprising:
identifying a set of devices associated with the location;
collecting data from each of the set of devices;
analyzing, via an application, the collected data;
determining, via the application, a set of patterns of activity for the user; and
storing, in a database, the set of patterns of activity, wherein the at least one pattern of activity is identified from the stored determined pattern of activity.

9. The method of claim 8, wherein the set of devices corresponds to devices comprising sensor capabilities for tracking movements and information about the user at the location.

10. The method of claim 1, wherein the device is a smart ring.

11. A device comprising:
a processor configured to:
collect data about a user, the user data comprising information related to activities of the user;
analyze the user data based on at least one pattern of activity of the user;
determine a sleep optimization recommendation for the user, the sleep optimization recommendation comprising a set of instructions to be performed within a location of the user prior to the user going to sleep; and
communicate, over a network, a notification to the user, the notification comprising information related to the sleep optimization recommendation, the notification comprising executable operations for managing the sleep of the user at the location.

12. The device of claim 11, wherein the notification causes automatic control of electronic devices at the location, the automatic control causing the electronic devices to operate in a manner as directed by the set of instructions.

13. The device of claim 11, wherein the notification is visibly displayed upon communication, wherein the visible display provides visibly displayed indications for the user related to how the user is recommended to act based on the set of instructions.

14. The device of claim 11, wherein the notification corresponds to real-world and digital components of an environment of the location, the environment corresponding to a bedroom of the user at the location.

15. The device of claim 11, wherein the processor is further configured to:
determining a time proximate the user going to sleep, the time proximate corresponding to a time period prior to the user sleeping at the location for which the set of instructions are to be performed, wherein the communication of the notification corresponds to the time proximate.

16. A non-transitory computer-readable storage medium tangibly encoded with computer-executable instructions that when executed by a device, perform a method comprising:
collecting, by the device, data about a user, the user data comprising information related to activities of the user;
analyzing, by the device, the user data based on at least one pattern of activity of the user;
determining, by the device, a sleep optimization recommendation for the user, the sleep optimization recommendation comprising a set of instructions to be performed within a location of the user prior to the user going to sleep; and
communicating, over a network, a notification to the user, the notification comprising information related to the sleep optimization recommendation, the notification comprising executable operations for managing the sleep of the user at the location.

17. The non-transitory computer-readable storage medium of claim 16, wherein the notification causes automatic control of electronic devices at the location, the automatic control causing the electronic devices to operate in a manner as directed by the set of instructions.

18. The non-transitory computer-readable storage medium of claim 16, wherein the notification is visibly displayed upon communication, wherein the visible display provides visibly displayed indications for the user related to how the user is recommended to act based on the set of instructions.

19. The non-transitory computer-readable storage medium of claim 16, wherein the notification corresponds to real-world and digital components of an environment of the location, the environment corresponding to a bedroom of the user at the location.

20. The non-transitory computer-readable storage medium of claim 16, further comprising:
determining a time proximate the user going to sleep, the time proximate corresponding to a time period prior to the user sleeping at the location for which the set of instructions are to be performed, wherein the communication of the notification corresponds to the time proximate.
